(19) United States
(12) Patent Application Publication
(10) Pub. No.: US 2014/0114053 A1
(43) Pub. Date: Apr. 24, 2014

(54) Electrophoresis Separation Methods

(71) Applicants: Ben Herbert, North Epping (AU); Andrew Arthur Gooley, North Epping (AU); Keith Leslie Williams, Frenchs Forest (AU)

(72) Inventors: Ben Herbert, North Epping (AU); Andrew Arthur Gooley, North Epping (AU); Keith Leslie Williams, Frenchs Forest (AU)

(21) Appl. No.: 14/135,578

(22) Filed: Dec. 20, 2013

Related U.S. Application Data
(63) Continuation of application No. 11/711,808, filed on Feb. 28, 2007, now abandoned.
Application No. 11/711,808 is a continuation of application No. 10/430,423, filed on May 7, 2003, now abandoned.
Application No. 10/430,423 is a continuation of application No. 09/319,450, filed on Aug. 27, 1999, now abandoned.

(30) Foreign Application Priority Data
Dec. 4, 1997 (AU) PCT/AU1997/970082

(51) Int. Cl. C07K 1/28

(52) U.S. Cl. CPC C07K 1/285
USPC 530/357; 204/548; 204/459

(57) ABSTRACT

An improved method of separating a macromolecule by isoelectric focusing comprising subjecting the macromolecule to electrophoresis in an isoelectric-focusing medium including a substantially thiol-free reducing agent, preferably a trivalent phosphorous compound and more preferably tributyl phosphine, the improvement being the solubility and focusing of the macromolecule is enhanced compared to isoelectric focusing of the same macromolecule in a similar isoelectric-focusing medium containing a thiol-reducing agent.

### TECHNICAL FIELD

The present invention relates to the field of gel electrophoresis and, particularly, to improved separation and gels for two-dimensional polyacrylamide gel electrophoresis.

### BACKGROUND ART

Two-dimensional polyacrylamide gel electrophoresis (2D-PAGE) has come into widespread use since the publication, in the early seventies, of methods combining isoelectric focusing (IEF) in the first dimension and sodium dodecyl sulphate polyacrylamide gel electrophoresis (SDS-PAGE) in the second dimension. Although 2D-PAGE provides the high resolution separations, preparative protein loads are difficult to achieve using conventional carrier ampholyte IEF (CA-IEF). Carrier ampholyte generated pH gradients are not fixed in the gel, and as a result, the gradients are prone to disruption. The main problems associated with CA-IEF are gradient drift and low buffering power, which lead to poor reproducibility and low protein capacity. In CA-IEF the pH gradient drift often causes the gradient to breakdown before all of the proteins in the sample reach steady state focusing positions. The introduction of immobilised pH gradients (IPGs) has solved the problems associated with CA-IEF, and made 2D-PAGE the method of choice for the preparative purification of proteins for analyses such as Edman sequencing, amino acid analysis and mass spectrometry [1, 2].

Poor transfer of protein from IPGs to the second dimension gel has been reported [3] and recently losses have been reported when membrane proteins were separated by 2D-PAGE using IPGs [4]. These losses have been attributed to protein adsorption to the IPG matrix at or near its isoelectric point, and they were not observed when IEF using carrier ampholytes were used for the first dimension [4]. The adsorption of proteins to the IPG matrix is probably due to hydrophobic interactions with the acrylamido buffering groups. A recent report showed that protein streaking in IPGs is directly related to the level of the hydrophobic pK 7.0 acrylamido buffer [5].

Hydrophobic interactions between proteins and the acrylamido buffers may occur during the prolonged focusing required to bring the proteins to a pH where they have zero net charge, and thus, reach a steady state. In addition, the protein insolubility observed in IPGs may be partly caused by the loss of dithiothreitol (DTT) during the very long run-times required for optimal focusing in IPGs, compared to CA-IEF The thiol groups on DTT will be ionised during IEF, which will cause transport of the DTT to the electrodes. When the DTT concentration drops during the IEF some proteins will become less soluble, as a result of the reformation of inter-chain disulphide bonds. After IEF in IPGs, to increase the solubility of the focused proteins and facilitate transfer to the second dimension gel, IPG strips are normally equilibrated for between 10 and 15 minutes in a solution of 1 or 2% DTT, 6M urea, 2% SDS, 20% glycerol and tris buffer at pH 6.8 [3]. The chaotropic action of urea in combination with the SDS will break the hydrophobic interactions between the proteins and the IPG matrix. High concentrations of DTT are required to re-solubilise proteins which may have re-crosslinked in the IPG.

To obtain correct SDS binding it is essential that the proteins are unfolded and all disulphide bonds are broken. A second equilibration step is done and DTT is replaced with between 2 and 4% iodoacetamide, which alkylates the free thiol groups and thus removes the excess DTT. The removal of the excess free thiols is desirable as the presence of free thiols, such as DTT, in the second dimension gel causes vertical streaking of the proteins and contributes to high background with silver staining.

In addition to equilibration in DTT, another approach to increasing protein solubility and transfer to the second dimension is to incorporate thiourea in the denaturing solution used for IEF in IPGs. The use of mixtures of urea, thiourea and surfactants such as CHAPS or SB 3-10 in the IPG was found to give increased protein solubility with samples that are prone to aggregation [4]. High concentrations of chaotropes such as thiourea, however, inhibit SDS binding to proteins, so thiourea cannot be used in the equilibration, and the maximum concentration of thiourea used in the IPG was 2M. Higher concentrations of thiourea caused vertical streaking, probably because the thiourea does not completely diffuse out of the IPG during the equilibration [4].

An additional problem with the current 2D-PAGE methodology, which is not addressed by the use of thiourea, or equilibration in DTT, is the formation of mixed adducts of cysteine arising from alkylation with iodoacetamide and acrylamide. It is unclear to what extent cysteine is alkylated with iodoacetamide during the equilibration. Gorg et al [3] reported that under the conditions of equilibration iodoacetamide reacts with the excess thiol-reducing agent without alkylating proteins. To avoid protein modification, however, Bjellqvist et al [6] eliminated the iodoacetamide in the equilibration when proteins from the 2D gel were to be used for antibody production. It is apparent that complete protein alkylation with iodoacetamide does not occur during the equilibration because acrylamide adducts of cysteine are normally observed during Edman sequencing and amino acid analysis (unpublished observations). Alkylation of proteins with acrylamide monomer occurs during the second dimension gel run, even after overnight polymerisation of the gel.

The formation of mixed adducts presents a number of problems during post-separation analysis. Many post-separation strategies for protein characterisation are based on mass spectrometry (MS) of the intact protein or peptide fragments, where it is advantageous to know what adducts may have been formed. Prior to enzymatic digestion it is important to block disulphide bond formation by reduction and alkylation, to simplify the peptide maps obtained. Moritz et al [7] have reported a reduction and alkylation protocol with DTT and 4-vinylpyridine, which is performed on a whole 1D or 2D gel, after Coomassie Brilliant Blue staining. In-situ tryptic cleavage of reduced and alkylated proteins was performed and the peptides were recovered and analysed by reversed phase high performance liquid chromatography (RP-HPLC) with on-line electrospray tandem MS. Cysteine containing peptides were identified during RP-HPLC by their characteristic absorbance at 254 nm and the appearance of a pyridylethyl fragment ion of 106 Da during electrospray tandem MS [7]. The alkylation of cysteine with 4-vinylpyridine after 2D electrophoresis indicates that complete alkylation with acrylamide monomer does not occur during the second dimension gel run. It would be impossible to alkylate, post-2D, with 4-vinylpyridine if complete alkylation had occurred during the equilibration and second dimension gel run. Therefore, it is probable that the procedure of Mortiz et al [7] results in the formation of three adducts of cysteine in some proteins, ie cys-iodoacetamide, cys-acrylamide and cys-vinylpyridine. Proteins which have formed more than one adduct of cysteine will be difficult to analyse using mass spectrometry, because it will not be possible to assume that every cysteine has had the same mass added to it.

In addition to mass spectrometry, amino acid composition matching and Edman ‘Tag’ sequencing can be used to rapidly screen and identify proteins separated by 2D-PAGE [8]. In Edman sequencing, non-alkylated cysteine residues are not recovered and a residue cannot be assigned at these positions in a sequence. In contrast, the PTH derivative of acrylamide alkylated cysteine is recovered and identified during the sequencing process. Likewise in amino acid analysis, the acrylamide adduct of cysteine is separated from the other amino acids and can be quantitated. This increases to 17 the number of amino acids which can he used for amino acid composition matching purposes.

In summary, although the use of IPGs in 2D-PAGE is a powerful technique for the preparative purification of proteins, a number of problems are inherent in the current methodology. The separated proteins are prone to adsorption to the IPG matrix in the first dimension separation, and high concentrations of DTT are required to give adequate transfer to the second dimension gel. In addition, the equilibration protocol currently used for solubilisation of proteins prior to transfer to the second dimension causes the formation of mixed adducts of cysteine, which complicates the post-separation analysis.

In order to address at least some of the problems associated with current methods used in electrophoresis, the present inventors have developed improved methods for the separation of macromolecules including polypeptides, proteins and glycoproteins by electrophoresis.

### DISCLOSURE OF INVENTION

In a first aspect, the present invention consists in a method of separating a macromolecule by isoelectric focusing comprising subjecting the macromolecule to electrophoresis in an isoelectric-focusing medium including a substantially thiol-free reducing agent.

The method according to the first aspect of the present invention offers an improvement in macromolecule separation over standard techniques of isoelectric focusing where thiol-reducing agents are used. The improvement being the solubility and focusing of the macromolecule is enhanced compared to isoelectric focusing of the same macromolecule in a similar isoelectric-focusing medium containing a thiol-reducing agent.

The thiol-free reducing agent increases the solubility and improves the focusing of the macromolecules over standard isoelectric focusing methods.

Preferably, the thiol-free reducing agent is a trivalent phosphorous compound, and more preferably tributyl phosphine (TBP). The concentration of the thiol-free reducing agent will vary depending on the amount and type of macromolecule being separated. Concentrations in the order of about 0.1 to 200 mM, preferably about 1 to 10 mM, have been found to be suitable but it will be appreciated that higher or lower concentrations can also be used. Preferably, for gel isoelectric focusing, the trivalent phosphorous compound should have the following properties: able to be solubilised in aqueous solutions; non-charged at normal isoelectric focusing pH values; and not readily explosive or highly reactive. It will be appreciated, however, that a charged trivalent phosphorous compound may also be useful in isoelectric focusing.

Preferably, for gel electrophoresis, the trivalent phosphorous compound should have the following properties: able to be solubilised in aqueous solutions; charged at normal isoelectric focusing pH values; and not readily explosive or highly reactive. It will be appreciated, however, that a non-charged trivalent phosphorous compound may also be useful in gel electrophoresis.

Other examples of trivalent phosphorous compounds suitable for the present invention include tris(pentafluorophenyl)phosphine, 4-(dimethylamino)phenyl-diphenyl-phosphine, tris(4-fluorophenyl)phosphine, tri(o-toly)phosphine, diphenyl(methoxymethyl)phosphine oxide, tri(m-toly)phosphine, tri(p-toly)phosphine, triethyl phosphine, tris(diethylamino)phosphine, tris(dimethylamino)phosphine, and tris(2-carboxyethyl)phosphine. It will be appreciated, however, that other trivalent phosphorous compounds may also be suitable for the present invention.

In one preferred embodiment of the first aspect of the present invention, the focusing is carried out substantially in the absence of thiol-containing reducing agents like dithiothreitol (DTT) presently used in standard isoelectric focusing techniques. In a preferred form, DTT is replaced by a lower concentration of TBP in standard methods presently in use, i.e. 100 mM DTT is replaced by about 1 to 10 mM, preferably about 2 mM, TBP. It will be appreciated, however, that under some separation or focusing situations it will be desirable to include both thiol-free and thiol-containing reducing agents during IEF.

The first aspect of the present invention is suitable for any IEF where reduction of the macromolecules is required. In particular, the method is particularly suitable where IEF is used as the first dimension prior to a second dimension of PAGE or SDS-PAGE in 2D-PAGE separations.

The thiol-reducing agent can be used in solution or, alternatively, bound or immobilised to the electrophoresis medium or walls or surfaces of the apparatus in which the electrophoresis separation is to be carried out which are in contact or associated with the macromolecule to be separated.

In a second aspect, the present invention consists in an improved method to separate a macromolecule by two dimensional polyacrylamide gel electrophoresis (2D-PAGE) comprising:
(a) separating the macromolecule by isoelectric focusing in a first dimension gel according the first aspect of the present invention;
(b) optionally, equilibrating the first dimension gel containing the macromolecule separated by (a) in the presence of a thiol-free reducing agent and an alkylating agent such that any free thiols are removed and substantially no mixed adducts of cysteine are formed; and
(c) further separating the macromolecule by polyacrylamide gel electrophoresis.

One major advantage of alkylating subsequent to the first dimension separation (optional step (b)) is that the macromolecule has been separated by charge in the first dimension and thus the alkylation does not affect the first dimension separation. Preferably, the alkylating agent is acrylamide or a fluorescent agent. The fluorescent agent can be selected from haloacetly derivatives, maleimides, miscellaneous sulfhydryl reagents, or mixtures thereof. One particularly suitable fluorescent agent is maleimide fluorescein.

The concentration of the alkylating agent will vary depending on the amount and type of macromolecules being treated in (b). Concentrations of acrylamide in the order of about 0.1 to 5%, preferably about 2.5% (w/v), have been found to be suitable but it will be appreciated that higher or lower concentrations can also be used. Concentrations of the fluorescent agent in the order of about 0.01 to 20 mM, preferably about 0.25 mM, have been found to be suitable but it will be appreciated that higher or lower concentrations can also be used. The further advantage of using a fluorescent agent as the alkylating agent is that the macromolecules are labelled by the agent prior to separation in the second dimension. This assists in the visualisation of the separated macromolecules without the need of additional staining after separation.

Other examples of alkylating agents suitable for the optional equilibration step (b) include monomers in use as replacements for acrylamide in gels. Examples include vinyl pyridine, N-acryloylaminoethoxyethanol, acrylamido-N,N-diethoxyethanol, N-acryloyl-tris(hydromethyl)aminomethane, acrylamido sugars such as N-acryloyl (or methacryloyl)-1-amino-deoxy-D-glucitol or the corresponding D-xylitol derivative, and N,N-diethylacrylamide. It will be appreciated, however, that other alkylating agents may also be suitable for the present invention.

Other examples of fluorescent agents suitable for the optional equilibration step (b) include haloacetyl derivatives, maleimides and miscellaneous sulfhydryl reagents that are readily available from suppliers such as Molecular Probes, Inc. It will be appreciated, however, that other fluorescent agents may also be suitable for the present invention.

It is also preferable that the equilibration, if required, is carried out substantially in the absence of iodoacetamide presently used in standard 2D-PAGE methods.

The separations are carried out in any suitable electrophoresis apparatus using electric currents and protocols presently in use.

In a third aspect, the present invention consists in the use of a thiol-free reducing agent in electrophoresis separation of a macromolecule.

It will be appreciated that the present invention would be suitable in any separation method where reduction of a macromolecule is desirable.

These methods include, but are not limited to, SDS-PAGE, isoelectric focusing, capillary zone electrophoresis, preparative electrophoresis methods, and the like. The thiol-reducing agent can be used in solution or, alternatively, bound or immobilised to the electrophoresis medium or walls or surfaces of the apparatus in which the electrophoresis separation is to be carried out which are in contact or associated with the macromolecule to be separated.

Preferably, the thiol-free reducing agent is a trivalent phosphorous compound, and more preferably tributyl phosphine (TBP). Other examples of trivalent phosphorous compounds suitable for the present invention include tris(pentafluorophenyl)phosphine, 4-(dimethylamino)phenyl-diphenyl-phosphine, tris(4-fluorophenyl)phosphine, tri(o-toly)phosphine, diphenyl(methoxymethyl)phosphine oxide, tri(m-toly)phosphine, tri(p-toly)phosphine, triethyl phosphine, tris(diethylamino)phosphine, tris(dimethylamino)phosphine, and tris(2-carboxyethyl)phosphine. It will be appreciated, however, that other trivalent phosphorous compounds may also be suitable for the present invention.

In a fourth aspect, the present invention consists in one or more macromolecules separated by the first or second aspect of the present invention.

The present invention is suitable to separate any macromolecule, particularly biomolecules including proteins, peptides and glycoproteins.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

In order that the present invention may be more clearly understood, preferred forms will be described with reference to the following examples and drawings.

### BRIEF DESCRIPTION OF DRAWINGS

FIG. 1 shows silver stained 2-D gels of wool proteins. FIG. 1a was separated using Solution A; 8M urea, 4% CHAPS, 100 mM DTT, 0.5% pH 3-10 ampholytes and 40 mM Tris, approximately pH 9.5. FIG. 1b was separated using Solution B; 8M urea, 4% CHAPS, 2 mm TBP, 0.5% pH 3-10 ampholytes and 40 mM Tris, approximately pH 9.5. After IEF for a total of 30,000 Vh the IPG in FIG. 1a was equilibrated in 6 M urea, 20% glycerol, 2% SDS and 2% DTT, 0.375M Tris at pH 8.8 for 10 minutes and then a further 10 minutes in the same solution except that DTT was replaced with 2.5% iodoacetamide, to alkylate any free DTT. The IPG in FIG. 1b was equilibrated in 6M urea, 20% glycerol, 2% SDS and 5 mm TBP, 0.375M Tris at pH 8.8 for 20 minutes. The intermediate filament proteins are poorly resolved in FIG. 1a, especially the Type I intermediate filament proteins. In contrast, FIG. 1b shows improved separation of the intermediate filament proteins with the Type I intermediate filament proteins well resolved into at least 4 major strings of spots (arrowed).

FIG. 2 shows Coomassie brilliant blue R250 stained 2-D gel of the same extract of wool proteins as in FIG. 1, separated by IEF for a total of 30,000 Vh using Solution B; 8M urea, 4% CHAPS, 2 mm TBP, 0.5% pH 3-10 ampholytes and 40 mM Tris, approximately pH 9.5 and equilibrated in 6 M urea, 20% glycerol, 2% SDS and 5 mm TBP, 0.375M Tris at pH 8.8 for 20 minutes. The Type I intermediate filament proteins are separated into 4 strings of spots (arrowed), reflecting the four Type I intermediate filament protein genes. The Type II intermediate filament proteins are separated into 2 major strings of spots (arrowed).

FIG. 3 shows silver stained 2-D gels of 1×10⁶ Chinese Hamster ovary (CHO) cell proteins. FIG. 3a was separated using Solution A; 8M urea, 4% CHAPS, 100 mM DTT, 0.5% pH 3-10 ampholytes and 40 mM Tris, approximately pH 9.5. FIG. 3b was separated using Solution B; 8M urea, 4% CHAPS, 2 mm TBP, 0.5% pH 3-10 ampholytes and 40 mM Tris, approximately pH 9.5. After IEF for a total of 80,000 Vh the IPG in FIG. 3a was equilibrated in 6M urea, 20% glycerol, 2% SDS and 2% DTT, 0.375M Tris at pH 8.8 for 10 minutes and then a further 10 minutes in the same solution except that DTT was replaced with 2.5% iodoacetamide, to alkylate any free DTT. The IPG in FIG. 3b was equilibrated in 6M urea, 20% glycerol, 2% SDS and 5 mm TBP, 0.375M Tris at pH 8.8 for 20 minutes. In FIG. 3a there is considerable horizontal streaking, which may be a result of disulfide bond re-formation during the IEF. In FIG. 3b the horizontal streaking has been eliminated and more protein spots are visible than in FIG. 3a. The spots indicated with arrows have resolved into multiple strings of different apparent mass when separated using TBP in the IEF.

FIG. 4 shows silver stained 2-D gels of 2 pairs of foetal Mouse limb bud proteins. FIG. 4a was separated using a modified Solution A; 8M urea, 4% CHAPS, 10 mM DTT, 0.5% pH 3-10 ampholytes and 40 mM Tris, approximately pH 9.5, and FIG. 4b was separated using Solution A; 8M urea, 4% CHAPS, 100 mM DTT, 0.5% pH 3-10 ampholytes and 40 mM Tris, approximately pH 9.5. FIG. 4c was separated using Solution B; 8M urea, 4% CHAPS, 2 mm TBP, 0.5% pH 3-10 ampholytes and 40 mM Tris, approximately pH 9.5. After IEF for a total of 60,000 Vh the IPGs in FIGS. 4a and 4b were equilibrated in 6M urea, 20% glycerol, 2% SDS and 2% DTT, 0.375M Tris at pH 8.8 for 10 minutes and then a further 10 minutes in the same solution except that DTT was replaced with 2.5% iodoacetamide, to alkylate any free DTT. The IPG in FIG. 4c was equilibrated in 6M urea, 20% glycerol, 2% SDS and 5 mm TBP, 0.375M Tris at pH 8.8 for 20 minutes. In FIG. 4a the focusing is poor compared to that with 100 MM DTT or TBP. FIG. 4c, using TBP, shows a considerable increase in spot numbers over FIGS. 4a and 4b.

### MODES FOR CARRYING OUT THE INVENTION

To demonstrate the effectiveness of the present invention, DTT was replaced with TBP to increase the solubility of proteins during the IEF. In order to simplify the equilibration process the conventional two step equilibration presently used has been replaced with an optional one step protocol using TBP and acrylamide. DTT was replaced as the reducing agent for a variety of reasons. Disulphide bond breaking with thiol containing reagents such as DTT is achieved by an equilibrium displacement process using a large excess of free thiols. Because high concentrations of free thiols are required to shift the equilibrium in favour of breaking disulphide bonds, in an alkylation, the majority of the alkylating agent reacts with the thiol reducing agent. Thus, it can be difficult to obtain a molar excess of alkylating agent. In contrast to thiol reducing agents, the phosphine family of reducing agents bring about reduction by a stoichiometric process rather than an equilibrium displacement [9]. A major advantage of the mechanism of phosphine reduction is that because phosphines do not contain a thiol they cannot be alkylated, which leads to a simplified reduction and alkylation protocol.

### Materials and Methods

### Materials

Tributyl phosphine (97% v/v) was obtained from Fluka. Piperazine diacrylamide (PDA), acrylamide, urea, Tris, glycine, ammonium persulphate, TEMED, 3-[(3-cholamidopropyl)dimethylammonio]-1-propanesulfonate (CHAPS), dithiothreitol (DTT) and poly-vinylidene di-fluoride membrane (PVDF) were obtained from Bio-Rad (Herecules, Calif.). ‘Ondina’ medicinal grade paraffin oil was obtained from Shell. Endonuclease was obtained from Sigma. All other chemicals were AnalaR grade obtained from BDH. Immobiline DryStrips and Pharmalyte pH 3-10 ampholytes were from Pharmacia (Uppsala, Sweden).

### Tributyl Phosphine Stock Solution

The TBP was made up as a 200 mM stock solution in anhydrous isopropanol. The TBP concentrate and the 200 mM stock solution were flushed with nitrogen after each use and stored at 4° C. This procedure should be done in a fume cupboard and other appropriate safety precautions such as gloves and laboratory coat should be worn. Concentrated TBP gives off noxious fumes on contact with dry organic material such as paper. Spills should be wiped up using a wet cloth.

### Isoelectric Focusing Sample Solutions

Two solutions were used to solubilise samples for IEF. Solution A was 8M urea, 4% CHAPS, 100 mM DTT, 0.5% pH 3-10 ampholytes and 40 mM Tris, approximately pH 9.5 not adjusted. For the Mouse limb buds an additional modified solution A was used, containing 10 mM DTT. Solution B was 8M urea, 4% CHAPS, 2 mm TBP, 0.5% pH 3-10 ampholytes and 40 mM Tris, approximately pH 9.5 not adjusted.

### Wool Protein Extraction

Wool from a Romney sheep was prepared and extracted according to the method of Herbert et al [10]. After extraction the supernatant was dialysed against five changes of deionised water and freeze dried. The extracted proteins were not alkylated. In preparation for IEF, 1 mg of freeze dried wool proteins was solubilised in solution A and 1 mg in solution B.

### Solubilisation of Chinese Hamster Ovary Cell Proteins

CHO K1 cells (1×10⁶ cells) were solubilised in 1 mL of solution A and 1×10⁶ cells in 1 mL of solution B. DNA was removed by the addition of endonuclease (150 units/mL) to the final solutions. The solutions were allowed to sit at room temperature for 1 hour before the IPG rehydration was started.

### Solubilisation of Foetal Mouse Limb Buds

Eight pairs of limb buds (13.5 days post-coitus) were solubilised in 2 mL of solution A and 8 pairs in 2 mL of solution B. DNA was removed by the addition of endonuclease (150 units/mL) to the final solutions. The solutions were allowed to sit at room temperature for 1 hour before the IPG rehydration was started.

### Isoelectric Focusing

For analytical and preparative gels, individual 18 cm Immobiline DryStrips, pH 4-7 or 3.5-10 non-linear, were rehydrated with 500 μL of protein solution in 2 mL plastic graduated pipettes cut to 19 cm long. Individual 11 cm pH 4-7 IPGs were rehydrated with 250 μL of protein solution in 2 mL plastic graduated pipettes cut to 12 cm long. Rehydration was allowed to proceed at room temperature for 24 hours. The IEF was carried out using a Pharmacia Multiphor II with a DryStrip Kit; power was supplied using a Consort 5000 V power supply and cooling water at 20° C. was supplied by a Pharmacia Multitemp III. The running conditions used for IEF with 11 cm and 18 cm IPGs were 300 V for 2 hours, 1000 V for 1 hour, 2500 V for 1 hour and a final phase of 5000 V up to a maximum of 80,000 Vh. The actual total Vh for each sample is given in each figure legend. After IEF the strips were stored at −80° C. until required for the second dimension.

### Equilibration of IPGs Using Dithiothreitol

IPGs which had been focused using solution A, containing DTT, were equilibrated using the conventional procedure. The IPGs were equilibrated in 6M urea, 20% glycerol, 2% SDS and 2% DTT, 0.375M Tris at pH 8.8 for 10 minutes and then a further 10 minutes in the same solution except that DTT was replaced with 2.5% iodoacetamide, to alkylate any free DTT. The equilibration solution was pH 8.8 because the second dimension gels do not have a stacking gel at pH 6.8 as is the case in the method of Gorg et al [1].

### Equilibration of IPGs Using Tributyl Phosphine

IPGs which had been focused using solution B, containing TBP, were equilibrated in 6M urea, 20% glycerol, SDS and 5 mm TBP, 0.375M Tris at pH 8.8 for 20 minutes.

### Second Dimension SDS-PAGE

Second dimension gels were run using the Protean IIxi from Bio-Rad (Hercules, Calif.). The gels were 1.5 mm thick, 8-18% T pore gradients, and were crosslinked with PDA at 2.5%C. The gel and anode buffers were 0.375M Tris/HCl, pH 8.8. Cathode buffer was 192 mM glycine adjusted to pH 8.3 using Tris, 0.1% (w/v) SDS and 0.001% (w/v) Bromophenol blue. The equilibrated IPG strips were embedded on the top of the SDS-PAGE gels using molten 1% (w/v) agarose in cathode buffer. Gels were run at constant current of 4 mA per gel for 2 hours and then 18 mA per gel overnight, until the Bromophenol blue front had traversed the gel.

The completed-analytical 2-D gels were stained with an ammoniacal silver stain. The preparative 2-D gels were stained overnight in 0.2% (w/v) Coomassie Brilliant Blue R250 in 30% (v/v) methanol, 5% (v/v) acetic acid. Destaining was in 30% (v/v) methanol.

### Results and Discussion

### Wool Protein Separation

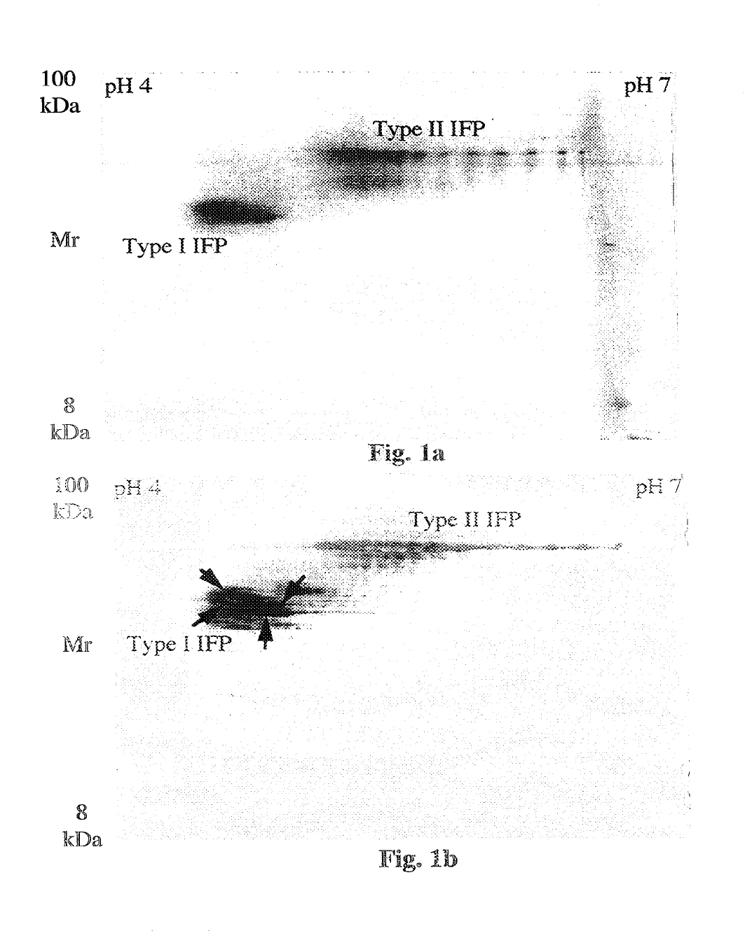

To investigate the ability of TBP to increase the solubility of proteins during IEF, the standard DTT protocol was compared to the TBP protocol using wool proteins. Wool is composed of two classes of proteins, the Intermediate Filament Proteins (IFP) and the Intermediate Filament Associated Proteins (IFAP). There are two sub-classes of IFP, Type I and Type II, and molecular biology and protein chemistry studies indicate that each subclass contains 4 structurally homologous proteins. The IFPs have been the subject of considerable study and are known to be post-translationally modified by glycosylation and phosphorylation. Herbert et al [10] obtained preparative separations of wool IFPs using DTT in the IEF, but the resolution of the IFPs was poor and it was not possible to separate individual IFP isoforms. By replacing the DTT in the IEF with TBP the separation is improved and it is possible to resolve the IFPs into individual spots. FIG. 1 shows silver stained 2-D gels of 150 μg of wool proteins separated under identical conditions except that FIG. 1a was separated in the first dimension using solution A, containing 100 mM DTT, and FIG. 1b was separated in the first dimension using solution B, containing 2 mm TBP. In FIG. 1a the IPG equilibration step was a conventional two-step process using firstly 2% DTT and secondly 2.5% iodoacetamide, and in FIG. 1b the IPG equilibration step was a single step process using 5 mm TBP. In FIG. 1a, using DTT in the IEF and equilibration, the IFPs are poorly resolved, especially the Type I IFP group. Using DTT in the IEF and equilibration, the resolution of the IFPs does not improve even after prolonged focusing at 5000 V for up to 500,000 Vh. Using TBP in the IEF and equilibration has improved the focusing and the IFPs are well resolved into at least 4 of strings of spots, (FIG. 1b). The proteins have reached steady state positions after 30,000 Vh, an IEF run time of 9 hours, which is a considerable improvement over almost 100 hours used for wool proteins previously.

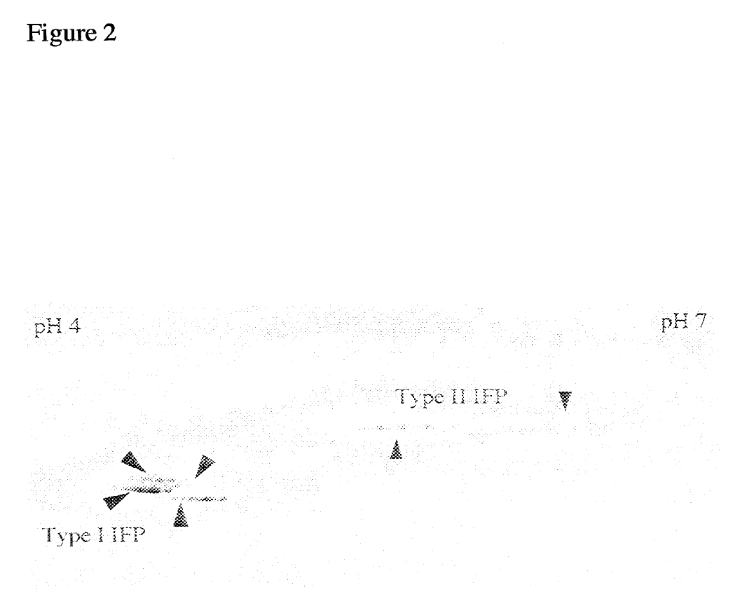

FIG. 2 is a Coomassie brilliant blue R250 stained preparative gel of 1 mg of the same extract of wool proteins as FIG. 1. The image has been cropped to show just the IFPs. The Type I IFPs are resolved into four strings, each containing at least 3 isoforms. The four gene products of the Type II IFPs are less clearly resolved than is the case for the Type I IFPs. Nevertheless, the Type II IFPs are resolved into two major strings of spots and some faintly stained strings of spots were visible on the gel close to the Type II IFPs, although, these do not appear clearly on the scanned image. Using the separation technology according to the present invention, it is now possible to quantitate the relative amounts of each of the Type I IFP and Type II gene products and their post-translational modifications. The ability to separate and quantitate the individual IFP gene products and their post-translational modifications is important in studying the role of IFPs in determining fibre properties such as strength and colour. Chinese Hamster ovary cell separation

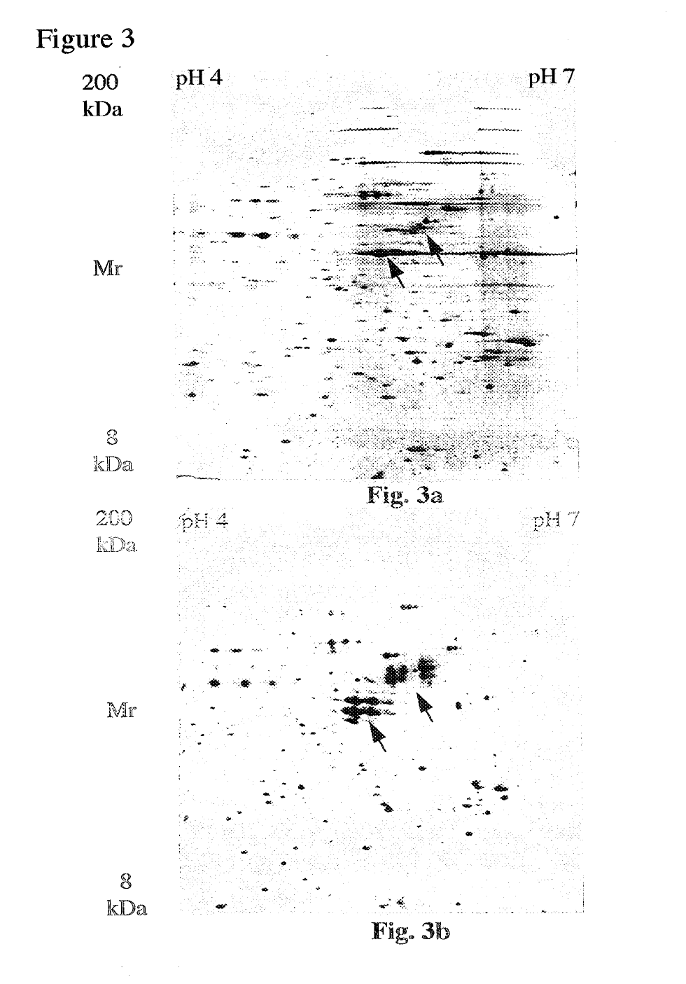

FIG. 3 shows silver stained 2-D gels of 1×10⁶ CHO K1 cells separated under identical conditions except that FIG. 3a was separated in the first dimension using solution A, containing 100 mm DTT, and FIG. 3b was separated in the first dimension using solution B, containing 2 mm TBP. In FIG. 3a, the IPG equilibration step was a conventional two-step process using firstly 2% DTT and secondly 2.5% iodoacetamide, and in FIG. 3b the IPG equilibration step was a single step process using 5 mm TBP. The horizontal streaking which is observed in FIG. 3a appears to be a result of incomplete focusing, which may indicate that some proteins have become insoluble during the IEF run. The horizontal streaking has been eliminated in FIG. 3b, which indicates that the proteins are more soluble using TBP and less prone to aggregate during the IEF. The increased solubility may be a result of the proteins being maintained in reducing conditions during the IEF and not re-forming inter-chain or intra-chain disulfide bonds. Some groups of spots (arrowed on FIGS. 3a and 3b) are resolved into multiple strings of different apparent mass in FIG. 3b, in contrast to FIG. 3a where they were poorly focused or only resolved into a single string. In culture systems such as CHO cells, the growth conditions can influence the macroheterogeneity and microheterogeneity of oligosaccharides in glycoproteins. Work in progress suggests that the multiple strings observed in FIG. 3b may be the result of differentially glycosylated forms of the same protein and that some glycoforms are sparingly soluble during IEF using DTT and are not normally resolved.

### Foetal Mouse Limb Buds

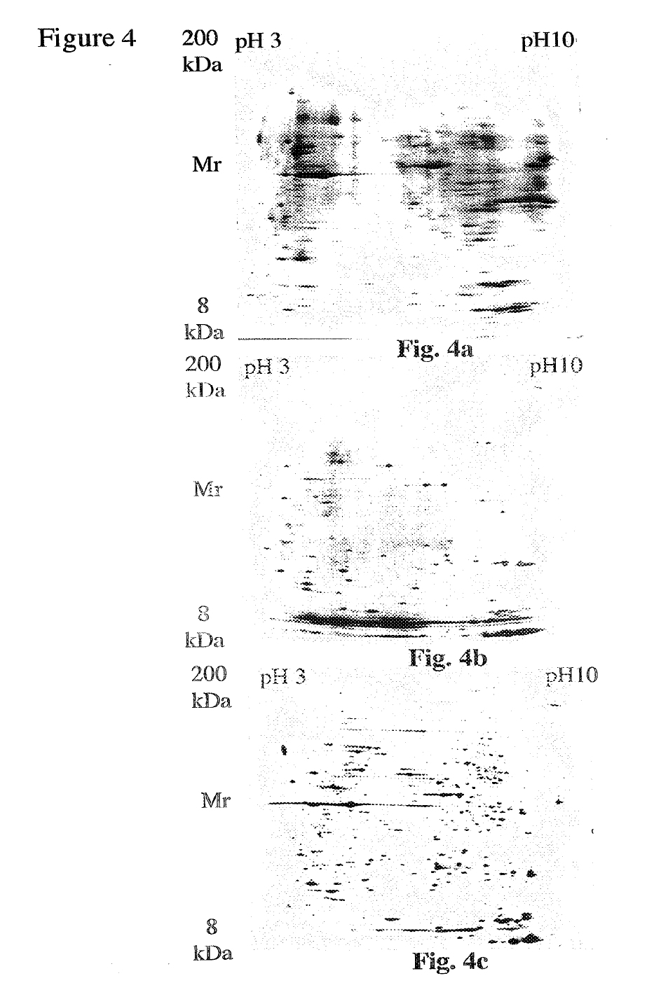

Many published IEF protocols use low concentrations of DTT, such as 10 mM to 20 mM, in the IPG rehydration solution. The present inventors were concerned that the highly streaky pattern obtained with CHO cells using 100 MM DTT in the IEF sample solution may be due to electroendosmosis resulting from the high concentration of charged DTT in the IPG. It was decided to expand this study using a mammalian tissue, which provides additional complexity compared to a cell line. Limb buds of 13.5 days post-coitus foetal mice were used as the model. FIG. 4 shows silver stained 2-D gels of 2 pairs of limb buds separated under identical conditions except that FIG. 4a was separated in the first dimension using a modified solution A, containing 10 mM DTT, FIG. 4b was separated in the first dimension using solution A, containing 100 MM DTT and FIG. 4c was separated in the first dimension using solution B, containing 2 mm TBP. In FIGS. 4a and 4b the IPG equilibration step was a conventional two-step process using firstly 2% DTT and secondly 2.5% iodoacetamide, and in FIG. 4c the IPG equilibration step was a single step process using 5 mm TBP. The separation achieved using TBP is superior to that with both 10 mM and 100 MM DTT. FIG. 4c has minimal horizontal and vertical streaking and the number of spots resolved is greater than in FIGS. 4a and 4b. The focusing is clearly worse in FIG. 4a where the DTT concentration is only 10 mM, which suggests that the DTT concentration is a parameter which should be optimised for each sample. Previously, 65 mM dithioerythritol (DTE) has been used in the IEF rehydration solution for micropreparative separations of yeast and liver samples without incurring problems with horizontal streaking. It has been stated that a typical reduction and alkylation experiment would require approximately 50 mM concentration of thiol reducing agents such as DTT to effect complete reduction of protein disulfides. Given that thiol reducing agents such as DTT act by equilibrium displacement, it seems unlikely that a concentration as low as 10 mM would be sufficient to force the equilibrium entirely to the formation of free thiols for the duration of an IEF run.

### Concluding Remarks

Improved separation in 2D PAGE gels has been demonstrated by replacing the thiol reducing agent DTT with tributyl phosphine. A major advantage of TBP is that it is not charged and thus does not migrate during the IEF. This means that the sample proteins are maintained under reducing conditions for the entire IEF process, which increases protein solubility and results in more protein spots on the final 2D PAGE gel. In most cases the use of TBP during the IEF results in a significant decrease in horizontal streaking.

The improved resolution obtained using TBP with both CHO K1 cells and the mouse limb buds suggests that it will be advantageous to use TBP for creating 2D maps of mammalian cells and tissues. The increased solubility and the ability to resolve differential glycoforms of proteins as observed in the 2D gels of CHO K1 cells (FIGS. 3a and 3b) will be essential in defining the complexity of mammalian cells and tissues.

A further advantage of TBP is that the IPG equilibration can be done in a single step because the uncharged TBP will not migrate and cause artifactual point streaking in the second dimension. In addition, it is possible to incorporate an alkylating agent, such as acrylamide or a fluorescent maleimide derivative, in the equilibration and thus alkylate cysteine residues on the separated proteins prior to the transfer to the second dimension gel.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### Abbreviations

IEF isoelectric focusing
SDS-PAGE sodium dodecyl sulphate polyacrylamide gel electrophoresis
2D-PAGE Two-dimensional polyacrylamide gel electrophoresis
CA-IEF carrier ampholyte isoelectric focusing
IPG immobilised pH gradient
MS mass spectrometry
RP-HPLC reversed phase high performance liquid chromatography
SDS sodium dodecyl sulphate
TBP tributyl phosphine
DTT dithiothreitol
IAA iodoacetamide
5IAF 5-iodoacetamido fluorescein
CHAPS 3-[(3-Cholamidopropyl)dimethylammonio]-1-propanesulfonate

### REFERENCES

1. Gorg, A., Postel, W. and Gunther, S. Electrophoresis. 1988, 9, 531-538.
2. Bjellqvist, B., Pasquali, C., Ravier, F., Sanchez, J-C. and Hochstrasser, D. F. Electrophoresis. 1993, 14, 1357-1365.
3. Gorg, A., Postel, W., Weser, J., Gunther, S., Strahler, J. R., Hanash, S. M. and Somerlot, L., Electrophoresis. 1987, 8, 122-124.
4. Rabilloud, T., Adessi, C., Giraudel, A. and Lunardi, J. Electrophoresis. 1997, 18, 307-316.
5. Esteve-Romero, J., Simo-Alfonso, E., Bossi, A., Bresciani, F. and Righetti, P. G. Electrophoresis. 1996, 17, 704-708.
6. Bjellqvist, B., Sanchez, J-C., Pasquali, C., Ravier, F., Paquet, N., Frutiger, S., Hughes, G. J. and Hochstrasser, D. F. Electrophoresis. 1993, 14, 1375-1378.
7. Moritz, R. L, Eddes, J. S., Reid, G. E. and Simpson, E. J. Electrophoresis. 1996, 17, 907-917.
8. Wilkins, M. R., Pasquali, C., Appel, R. D., Ou, K., Golaz, O., Sanchez, J-C., Yan, J. X., Gooley, A. A., Hughes, G., Humphery-Smith, I., Williams, K. L., Hochstrasser, D. F. Bio/Technology, 1996, 14, 61-65.
9. T. Rabilloud. Electrophoresis. 1996, 17, 813-829.
10. Herbert, B. R., Chapman, A. L. P. and Rankin, D. A. Electrophoresis. 1996, 17, 239-243.
11. Hughes, G. J., Frutiger, S., Paquet, N., Ravier, F., Pasquali, C., Sanchez, J-C., James, R., Tissot, J. D., Bjellqvist, B. and Hochstrasser, D. F. Electrophoresis. 1992, 13, 707-714.
12. Hochstrasser, D. F., Harrington, M. G., Hochstrasser, A. C., Miller, M. J. and Merril, C. R., Anal. Biochem., 1988, 173, 424-435.

1. A method of separating a macromolecule by isoelectric focusing comprising subjecting the macromolecule to electrophoresis in an isoelectric-focusing medium including a substantially thiol-free reducing agent.

2. The method according to claim 1 wherein the thiol-free reducing agent is a trivalent phosphorous compound.

3. The method according to claim 2 wherein the trivalent phosphorous compound is selected from the group consisting of tris(pentafluorophenyl)phosphine, 4-(dimethylamino)phenyl-diphenyl-phosphine, tris(4-fluorophenyl)phosphine, tri(o-toly)phosphine, diphenyl(methoxymethyl)phosphine oxide, trim-toly)phosphine, tri(p-toly)phosphine, triethyl phosphine, tris(diethylamino)phosphine, tris(dimethylamino)phosphine, tributyl phosphine, and tris(2-carboxyethyl)phosphine.

4. The method according to claim 3 wherein the trivalent phosphorous compound is tributyl phosphine.

5. The method according to any one of claims 1 to 4 wherein the concentration of the thiol-free reducing agent is 0.1 to 200 mM.

6. The method according to claim 5 wherein the concentration of the thiol-free reducing agent is 1 to 10 mM.

7. The method according to any one of claims 1 to 6 wherein the isoelectric focusing of the macromolecule is carried out substantially in the absence of thiol-containing reducing agents.

8. The method according to any one of claims 1 to 7 wherein the thiol-free reducing agent is in an immobilised form.

9. A method of separating a macromolecule by two dimensional polyacrylamide gel electrophoresis (2D-PAGE) comprising:
(a) separating the macromolecule by isoelectric focusing in a first dimension gel by subjecting the macromolecule to electrophoresis in an isoelectric-focusing medium including a substantially thiol-free reducing agent;
(b) optionally, equilibrating the macromolecule separated in the first dimension gel by (a) in the presence of a thiol-free reducing agent and an alkylating agent such that any free thiols are removed and substantially no mixed adducts of cysteine are formed; and
(c) further separating the macromolecule by polyacrylamide gel electrophoresis.

10. The method according to claim 9 wherein the thiol-free reducing agent is a trivalent phosphorous compound.

11. The method according to claim 10 wherein the trivalent phosphorous compound is selected from the group consisting of tris(pentafluorophenyl)phosphine, 4-(dimethylamino)phenyl-diphenyl-phosphine, tris(4-fluorophenyl)phosphine, tri(o-toly)phosphine, diphenyl(methoxymethyl)phosphine oxide, tri(m-toly)phosphine, tri(p-toly)phosphine, triethyl phosphine, tris(diethylamino)phosphine, tris(dimethylamino)phosphine, tributyl phosphine, and tris(2-carboxyethyl)phosphine.

12. The method according to claim 11 wherein the trivalent phosphorous compound is tributyl phosphine.

13. The method according to any one of claims 9 to 12 wherein the concentration of the thiol-free reducing agent concentration of the thiol-free reducing agent is 0.1 to 200 mM.

14. The method according to claim 13 wherein the concentration of the thiol-free reducing agent is 1 to 10 mM.

15. The method according to any one of claims 9 to 14 wherein the isoelectric focusing of the macromolecule in (a) is carried out substantially in the absence of thiol-containing reducing agents.

16. The method according to any one of claims 9 to 15 wherein the thiol-free reducing agent is in an immobilised form.

17. The method according to any one of claims 9 to 16 wherein the alkylating agent is selected from the group consisting of acrylamide, a fluorescent agent, N-acryloylaminoethoxyethanol, acrylamido-N,N-diethoxyethanol, N-acryloyl-tris(hydromethyl)aminomethane, acrylamido sugars such as N-acryloyl (or methacryloyl)-1-amino-deoxy-D-glucitol or the corresponding D-xylitol derivative, and N,N-diethylacrylamide.

18. The method according to claim 17 wherein the alkylating agent is acrylamide.

19. The method according to claim 18 wherein the concentration of the acrylamide is 0.1 to 5% (w/v).

20. The method according to claim 19 wherein the concentration of the acrylamide is 2.5% (w/v).

21. The method according to claim 17 wherein the fluorescent agent is selected from the group consisting of haloacetly derivatives, maleimides and miscellaneous sulfhydryl reagents.

22. The method according to claim 21 wherein the fluorescent agent is maleimide fluorescein.

23. The method according to claim 21 or 22 wherein the concentration of the fluorescent agent is 0.01 to 20 mM.

24. The method according to claim 23 wherein the concentration of the fluorescent agent is 0.25 mM.

25. The method according to any one of claims 9 to 24 wherein the optional equilibrating of the macromolecule in (b) is carried out substantially in the absence of iodoacetamide.

26. Use of a thiol-free reducing agent in an electrophoresis separation of a macromolecule.

27. The use according to claim 26 wherein the thiol-free reducing agent is a trivalent phosphorous compound.

28. The use according to claim 27 wherein the trivalent phosphorous compound is selected from the group consisting of tris(pentafluorophenyl)phosphine, 4-(dimethylamino)phenyl-diphenyl-phosphine, tris(4-fluorophenyl)phosphine, tri(o-toly)phosphine, diphenyl(methoxymethyl)phosphine oxide, trim-toly)phosphine, tri(p-toly)phosphine, triethyl phosphine, tris(diethylamino)phosphine, tris(dimethylamino)phosphine, tributyl phosphine, and tris(2-carboxyethyl)phosphine.

29. The use according to claim 28 wherein the trivalent phosphorous compound is tributyl phosphine.

30. The use according to any one of claims 26 to 29 wherein the thiol-free reducing agent is in an immobilised form.

31. A macromolecule separated by the method according to any one of claims 1 to 8.

32. A macromolecule separated by the method according to any one of claims 9 to 25.
